(19) United States
(12) Patent Application Publication
(10) Pub. No.: US 2002/0164704 A1
(43) Pub. Date: Nov. 7, 2002

(54) Novel human monocyte chemotactic proprotein

(75) Inventors: Roger Coleman, Mountain View, CA (US); Jennifer L. Hillman, San Jose, CA (US); Janice Au-Young, Berkeley, CA (US)

Correspondence Address: INCYTE GENOMICS, INC., 3160 PORTER DRIVE, PALO ALTO, CA, 94304, US

(73) Assignee: Incyte Pharmaceuticals, Inc.

(21) Appl. No.: 10/033,067

(22) Filed: Oct. 26, 2001

Related U.S. Application Data
(63) Continuation of application No. 08/683,655, filed on Jul. 15, 1996, now abandoned.

(51) Int. Cl. A01K 67/00; C07H 21/04; C12N 9/00; C12P 21/02; C12N 5/06; C12N 1/21

(52) U.S. Cl. 435/69.1; 435/183; 435/320.1; 435/325; 536/23.2; 435/252.3; 800/8

(57) ABSTRACT

The present invention provides a polynucleotide which identifies and encodes a novel human monocyte chemotactic proprotein (MCPP). The invention provides for genetically engineered expression vectors and host cells comprising the nucleic acid sequence encoding MCPP. The invention also provides for the use of substantially purified MCPP and its agonists, antibodies, antagonists or inhibitors in pharmaceutical compositions for treatment of diseases associated with expression of MCPP. Additionally, the invention provides for use of antisense molecules in pharmaceutical compositions for treatment of diseases associated with the expression of MCPP. The invention also describes diagnostic assays which utilize diagnostic compositions comprising the polynucleotide, fragments or the complement thereof, which hybridize with the genomic sequence or transcripts of MCPP and anti-MCPP antibodies which specifically bind to MCPP.

This application is a continuation application of U.S. application Ser. No. 08/683,655, filed Jul. 15, 1996, entitled NOVEL HUMAN MONOCYTE CHEMOTACTIC PROPROTEIN, which is hereby expressly incorporated by reference herein.

### FIELD OF THE INVENTION

The present invention relates to nucleic acid and amino acid sequences of a novel monocyte chemotactic proprotein and to the use of these sequences in the diagnosis, study, prevention and treatment of disease.

### BACKGROUND OF THE INVENTION

Monocyte chemotactic proteins are CC chemokines, small secreted polypeptides, generally about 70-100 amino acids in length, 8-11 kD in molecular weight, and active over a 1-100 ng/ml concentration range. The monocyte chemotactic proteins and closely related polypeptides are assigned to the CC chemokine family, display definitive spacing of the first two cysteine residues in the mature molecule, and act on a diverse group of target cells. Initially, the CC chemokines were isolated and purified from inflamed tissues and characterized relative to their bioactivity. More recently, these chemokines have been discovered through molecular cloning techniques and characterized by structural and functional analysis.

One function of the CC chemokines is to mediate the expression of particular selectin, integrin or other adhesion molecules on endothelial cells Their activity in this regard indirectly governs diapedesis and extravasation. They also generate gradients of chemoattractant factors which activate, and may cause the proliferation of, specific cell types such as monocytes, macrophages, basophils, eosinophils, T lymphocyte, and fibroblasts through specific cell surface receptors. The known chemokines and their functions are reviewed by Thomson (1994; The Cytokine Handbook, Academic Press, New York N.Y.) and Schall T J (1994) Chemotactic Cytokines: Targets for Therapeutic Development, International Business Communications, Southborough Mass., pp 180-270).

MCP-1, which is identical to hJE, is a 76 amino acid mature protein which appears to be expressed in almost all cells and tissues upon stimulation by a variety of agents. According to Charo I et al (Proc Natl Acad Sci USA 91:2752-2756), the targets of MCP-1 may be limited to monocytes and basophils in which it induces an mcp-1 receptor:G protein-linked calcium flux. Shyy Y-J et al (1990; Biochem Biophys Res Commun 169:346-351) reported that MCP-1 was induced in endothelial cell cultures by phorbol ester treatment and may be implicated in atherogenesis. MCP-2 and MCP-3 which have been purified from a human osteosarcoma cell line (Van Damme J et al (1993; Adv Exp Med Biol 351:111-118) have 62% and 73% amino acid identity, respectively, with MCP-1 and share its chemotactic specificity for monocytes. Minty A et al (1993; Eur Cytokine Netw 4:99-110) reported that recombinant MCP-3 is an N-glycosylated molecule expressed at lower levels than MCP-1. MCP-3 and MCP-1 mRNAs appear to be co-ordinately regulated in monocytes in response to a number of inducing agents including cycloheximide, interferon-gamma, and phorbol ester.

Understanding the structure and function of the monocyte chemotactic proteins provides opportunities to modulate those conditions associated with altered chemokine expression. Such conditions include inflammatory, infectious, autoimmune and hereditary diseases such as AIDS, asthma, carcinomas, and rheumatoid arthritis which involve the activation of monocytes and macrophages.

### SUMMARY OF THE INVENTION

The present invention discloses a novel human monocyte chemotactic proprotein, MCPP, characterized as having homology to MCP-2 and MCP-3. Accordingly, the invention features a substantially purified human monocyte chemotactic proprotein, as shown in amino acid sequence of SEQ ID NO:1, and having characteristics of the CC chemokines.

One aspect of the invention features isolated and substantially purified polynucleotides which encode MCPP. In a particular aspect, the polynucleotide is the nucleotide sequence of SEQ ID NO:2. In addition, the invention features polynucleotide sequences that hybridize under stringent conditions to SEQ ID NO:2.

A nucleic acid sequence encoding MCPP, oligonucleotides, peptide nucleic acids, fragments, portions or antisense molecules thereof, may be used in diagnostic assays of body fluids or biopsied tissues to detect the expression level of a nucleic acid sequence encoding MCPP. For example, a nucleic acid sequence designed from SEQ ID NO:2 can be used to detect the presence of the mRNA transcripts in a patient or to monitor modulation of such transcripts during treatment.

The present invention relates, in part, to the inclusion of the polynucleotide encoding MCPP in an expression vector which can be used to transform host cells or organisms. Such transgenic hosts are useful for the production of MCPP. Substantially purified MCPP, or fragments thereof, may be useful for treating autoimmune diseases such as AIDS.

A nucleic acid sequence encoding MCPP also provides for the design of antisense molecules useful in diminishing or eliminating expression of the genomic nucleotide sequence in conditions where MCPP may promote inappropriate monocyte or macrophage activity causing damage associated with allergic response to organs such as the lung.

The invention further provides diagnostic assays for the detection of naturally occurring MCPP. It provides for the use of substantially purified MCPP as a positive control and to produce anti-MCPP antibodies which can be used to quantitate the amount of MCPP in body fluids or biopsied tissues. MCPP can also be used to identify agonists, antagonists, or inhibitors to modulate the activity of MCPP in allergic responses or autoimmune diseases.

The invention also relates to pharmaceutical compositions comprising MCPP, antisense molecules capable of disrupting expression of the genomic sequence encoding MCPP, and agonists, antibodies, antagonists or inhibitors of the MCPP. These compositions are useful for the prevention or treatment of conditions associated with the expression of MCPP.

### DESCRIPTION OF THE FIGURES

FIGS. 1A and 1B displays the amino acid and nucleic acid sequences of the novel monocyte chemotactic proprotein produced using the multisequence alignment program of MACDNASIS software (Hitachi Software Engineering Co Ltd).

FIG. 2 shows the amino acid sequence similarity between MCPP (SEQ ID NO:1) and hJE-2 (GI 338809; SEQ ID NO:4), and MCP-3 (GI 288397; SEQ ID NO:3) produced using the multisequence alignment program of DNASTAR software (DNAStar Inc, Madison Wis.).

FIG. 3 shows the hydrophobicity plot for MCPP, SEQ ID NO:1, generated using MACDNASIS software; the X axis reflects amino acid position, and the negative Y axis, hydrophobicity.

FIG. 4 shows the isoelectric plot for MCPP, SEQ ID NO:1, generated using MacDNAsis software.

FIG. 5 shows the electronic northern analysis generated using BLAST and the LIFESEQ database (Incyte Pharmaceuticals, Palo Alto Calif.).

### DESCRIPTION OF THE INVENTION

### Definitions

“Nucleic acid sequence” as used herein refers to an oligonucleotide, nucleotide or polynucleotide, and fragments or portions thereof, and to DNA or RNA of genomic or synthetic origin which may be single- or double-stranded, and represent the sense or antisense strand. Similarly, amino acid sequence as used herein refers to an oligopeptide, peptide or protein sequence.

“Peptide nucleic acid” as used herein refers to a molecule which comprises an oligomer to which an amino acid residue, such as lysine, and an amino group have been added. These small molecules, also designated anti-gene agents, stop transcript elongation by binding to their complementary (template) strand of nucleic acid (Nielsen P E et al (1993) Anticancer Drug Des 8:53-63).

As used herein, MCPP refers to the amino acid sequence of substantially purified monocyte chemotactic proprotein from any source whether natural, synthetic, semi-synthetic or recombinant.

The present invention also encompasses MCPP variants. A preferred MCPP variant is one having at least 80% amino acid sequence similarity to the MCPP of SEQ ID NO:1, a more preferred MCPP variant is one having at least 90% sequence similarity to SEQ ID NO:1, and a most preferred MCPP variant is one having at least 95% sequence similarity to SEQ ID NO:1.

A “variant” of MCPP may have an amino acid sequence that is different by one or more amino acid “substitutions”. The variant may have “conservative” changes, wherein a substituted amino acid has similar structural or chemical properties, eg, replacement of leucine with isoleucine. More rarely, a variant may have “nonconservative” changes, eg, replacement of a glycine with a tryptophan. Similar variations may also include amino acid deletions or insertions, or both. Guidance in determining which and how many amino acid residues may be substituted, inserted or deleted without abolishing biological or immunological activity may be found using computer programs well known in the art, for example, DNASTAR software.

The term “biologically active” refers to a MCPP having structural, regulatory or biochemical features of the naturally occurring MCPP. Likewise, “immunologically active” defines the capability of the natural, recombinant or synthetic MCPP, or any oligopeptide thereof, to induce a specific immune response in appropriate animals or cells and to bind with specific antibodies.

The term “derivative” as used herein refers to the chemical modification of a nucleic acid sequence encoding MCPP or the encoded MCPP. Illustrative of such modifications would be replacement of hydrogen by an alkyl, acyl, or amino group. A nucleic acid derivative would encode a polypeptide which retains essential biological characteristics of natural MCPP.

As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated.

### Description

The present invention relates to a novel human monocyte chemotactic proprotein, MCPP, initially identified among the partial cDNAs from a breast library (BRSTNOT05) and to the use of the nucleic acid and amino acid sequences disclosed herein in the study, diagnosis, prevention and treatment of disease.

Nucleic acid sequence encoding a portion of MCPP was first identified in the CDNA, Incyte Clone 965517 through a computer-generated search for amino acid sequence alignments. Although nucleotide sequences encoding all or part of MCPP were also found in bladder tumor, lymphocyte, lung, macrophage, NIDDM pancreas, and rheumatoid synovium libraries, the naturally occurring expression is not necessarily limited to these cells or tissues. The nucleic acid sequence disclosed herein was assembled using Incyte Clones 965517 (SEQ ID NO:2), 515733 (SEQ ID NO:5), 518226 (SEQ ID NO:6), and 568961 (SEQ ID NO:7; and it encodes the amino acid sequence for MCPP, SEQ ID NO:1.

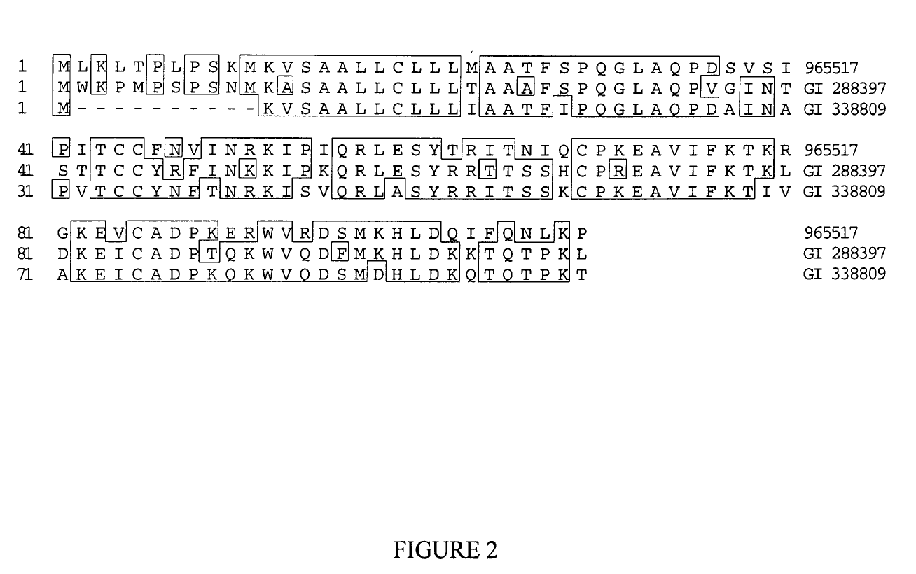

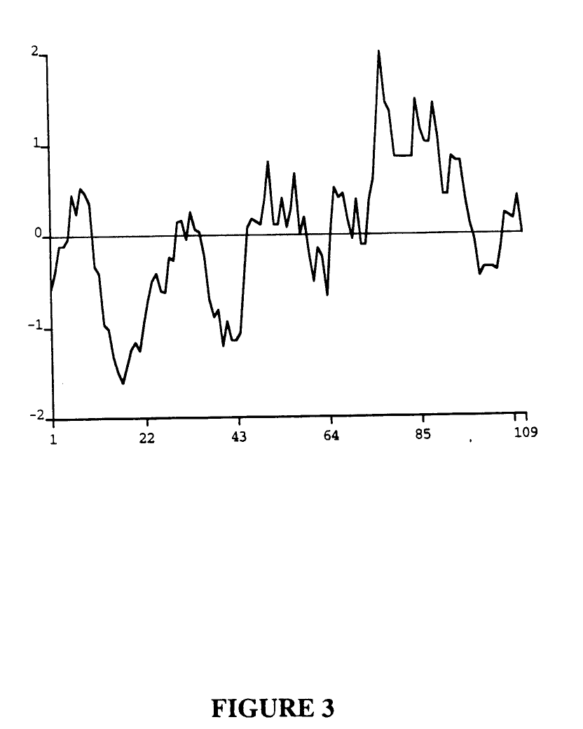

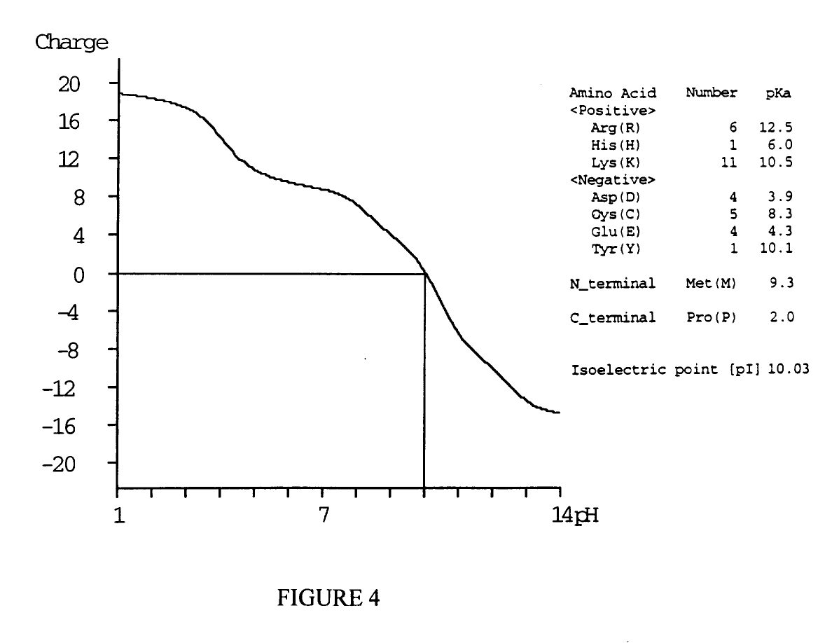

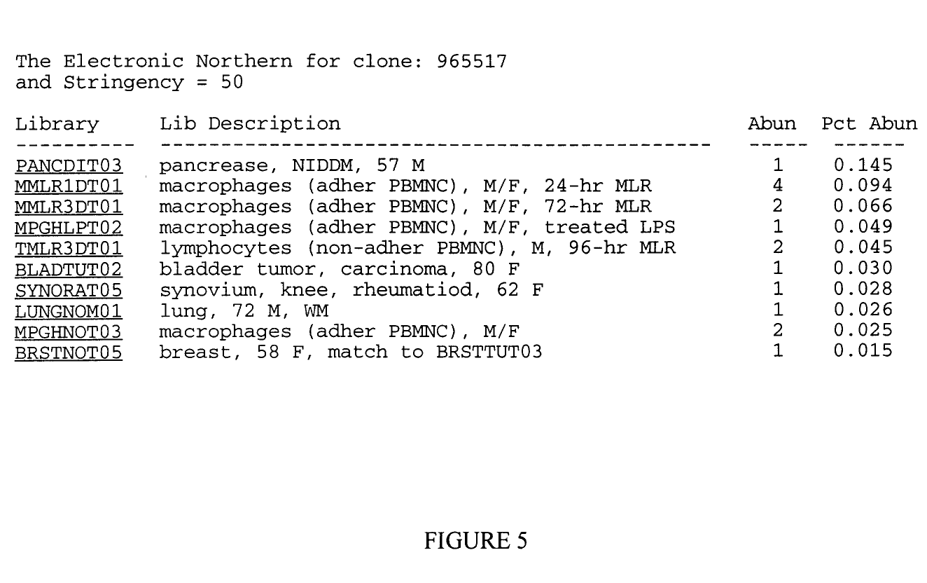

The present invention is based, in part, on the chemical and structural homology among MCPP, hJE-2 (GI 338809; Rollins B J et al (1989) Mol Cell Biol 9:4687-4695), and MCP-3 (GI 288397; Minty, supra). MCPP has 109 amino acids and 64% amino acid sequence identity to MCP-1; 63%, to hJE/MCP-2; and 62%, to MCP-3. AS shown in FIG. 2, MCPP has the conserved and definitive cysteine residues of the CC chemokines at positions, C₁₉, C₄₄, C₄₅, C₆₉ and C_{85;} however, it does not share the N₄₇ glycosylation site common to the other three molecules. The hydrophobicity and isoelectric plots of the secreted MCPP dislosed herein are shown in FIGS. 4 and 5.

### The MCPP Coding Sequences

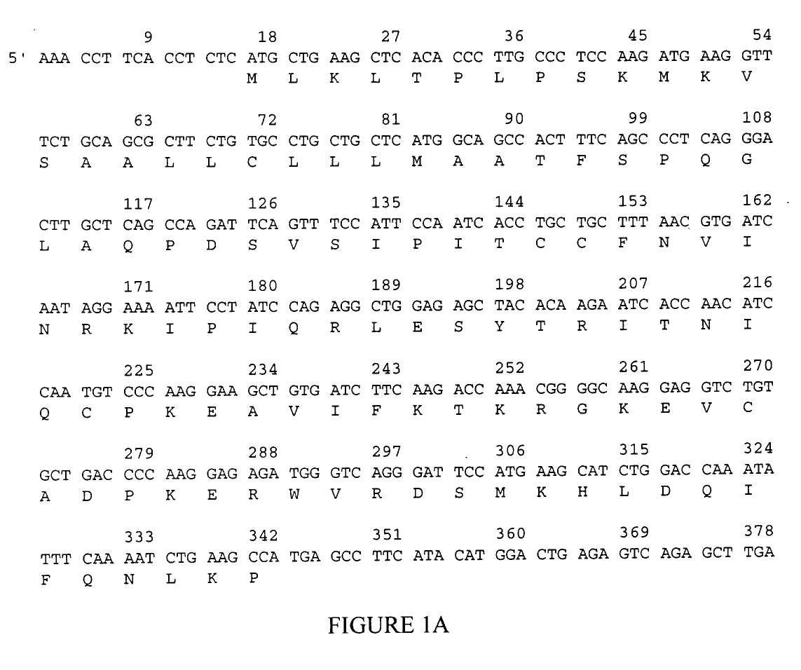

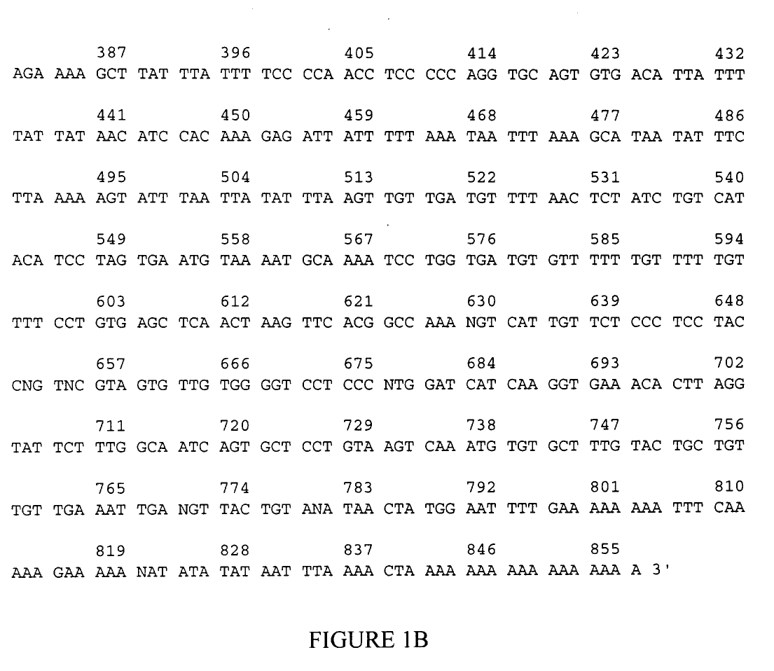

The nucleic acid and deduced amino acid sequences of MCPP are shown in FIGS. 1A and 1B. In accordance with the invention, any nucleic acid sequence which encodes the amino acid sequence of MCPP can be used to generate recombinant molecules which express MCPP. In a specific embodiment described herein, the sequence of MCPP was first identified in Incyte Clone 965517 from a breast cDNA library (BRSTNOT05).

It will be appreciated by those skilled in the art that, as a result of the degeneracy of the genetic code, a multitude of MCPP-encoding nucleotide sequences, some bearing minimal homology to the nucleotide sequences of any known and naturally occurring gene may be produced. The invention has specifically contemplated each and every possible variation of nucleotide sequence that could be made by selecting combinations based on possible codon choices. These combinations are made in accordance with the standard triplet genetic code as applied to the nucleotide sequence of naturally occurring MCPP, and all such variations are to be considered as being specifically disclosed.

Although nucleotide sequences which encode MCPP and its variants are preferably capable of hybridizing to the nucleotide sequence of the naturally occurring MCPP under appropriately selected conditions of stringency, it may be advantageous to produce nucleotide sequences encoding MCPP or its derivatives possessing a substantially different codon usage. Codons may be selected to increase the rate at which expression of the peptide occurs in a particular prokaryotic or eukaryotic expression host in accordance with the frequency with which particular codons are utilized by the host. Other reasons for substantially altering the nucleotide sequence encoding MCPP and its derivatives without altering the encoded amino acid sequences include the production of RNA transcripts having more desirable properties, such as a greater half-life, than transcripts produced from the naturally occurring sequence.

It is now possible to produce a DNA sequence, or portions thereof, encoding a MCPP and its derivatives entirely by synthetic chemistry, after which the synthetic gene may be inserted into any of the many available DNA vectors and cell systems using reagents that are well known in the art at the time of the filing of this application. Moreover, synthetic chemistry may be used to introduce mutations into a MCPP sequence or any portion thereof.

Also included within the scope of the present invention are polynucleotide sequences that are capable of hybridizing to the nucleotide sequence of Figs SEQ ID NO:2 under various conditions of stringency. Hybridization conditions are based on the melting temperature (Tm) of the nucleic acid binding complex or probe, as taught in Berger and Kimmel (1987, “Guide to Molecular Cloning Techniques”, Methods in Enzymolocy, Vol 152, Academic Press, San Diego Calif.) incorporated herein by reference, and confer may be used at a defined “stringency” as explained below.

“Maximum stringency” typically occurs at about Tm-5° C. (5° C. below the Tm of the probe); “high stringency” at about 5° C. to 10° C. below Tm; “intermediate stringency” at about 10° C. to 20° C. below Tm; and “low stringency” at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

The term “hybridization” as used herein shall include “any process by which a strand of nucleic acid joins with a complementary strand through base pairing” (Coombs J (1994) Dictionary of Biotechnology, Stockton Press, New York N.Y.). Amplification as carried out in polymerase chain reaction technologies is described in Dieffenbach C W and G S Dveksler (1995, PCR Primer, a Laboratory Manual, Cold Spring Harbor Press, Plainview N.Y.).

A “deletion” is defined as a change in either nucleotide or amino acid sequence in which one or more nucleotides or amino acid residues, respectively, are absent.

An “insertion” or “addition” is that change in a nucleotide or amino acid sequence which has resulted in the addition of one or more nucleotides or amino acid residues, respectively, as compared to the naturally occurring MCPP or MCPP.

A “substitution” results from the replacement of one or more nucleotides or amino acids by different nucleotides or amino acids, respectively.

Altered MCPP nucleic acid sequences which may be used in accordance with the invention include deletions, insertions or substitutions of different nucleotides resulting in a polynucleotide that encodes the same or a functionally equivalent MCPP. The protein may also show deletions, insertions or substitutions of amino acid residues which produce a silent change and result in a functionally equivalent MCPP. Deliberate amino acid substitutions may be made on the basis of similarity in polarity, charge, solubility, hydrophobicity, hydrophilicity, and/or the amphipathic nature of the residues as long as the biological activity of MCPP is retained. For example, negatively charged amino acids include aspartic acid and glutamic acid; positively charged amino acids include lysine and arginine; and amino acids with uncharged polar head groups having similar hydrophilicity values include leucine, isoleucine, valine; glycine, alanine; asparagine, glutamine; serine, threonine phenylalanine, and tyrosine.

Included within the scope of the present invention are alleles of MCPP. As used herein, an “allele” or “allelic sequence” is an alternative form of MCPP. Alleles result from a mutation, ie, a change in the nucleic acid sequence, and generally produce altered mRNAs or polypeptides whose structure or function may or may not be altered. Any given gene may have none, one or many allelic forms. Common mutational changes which give rise to alleles are generally ascribed to natural deletions, additions or substitutions of amino acids. Each of these types of changes may occur alone, or in combination with the others, one or more times in a given sequence.

Methods for DNA sequencing are well known in the art and employ such enzymes as the Klenow fragment of DNA polymerase I, SEQUENASE (US Biochemical Corp, Cleveland Ohio), Taq polymerase (Perkin Elmer, Norwalk Conn.), thermostable T7 polymerase (Amersham, Chicago Ill.), or combinations of recombinant polymerases and proofreading exonucleases such as the ELONGASE Amplification System marketed by Gibco BRL (Gaithersburg Md.). Preferably, the process is automated with machines such as the Hamilton Micro Lab 2200 (Hamilton, Reno Nev.), Peltier Thermal Cycler (PTC200; MJ Research, Watertown Mass.) and the ABI 377 DNA sequencers (Perkin Elmer).

### Extending the Polynucleotide Sequence

The polynucleotide sequence of MCPP may be extended utilizing partial nucleotide sequence and various methods known in the art to detect upstream sequences such as promoters and regulatory elements. Gobinda et al (1993; PCR Methods Applic 2:318-322) disclose “restriction-site” polymerase chain reaction (PCR) as a direct method which uses universal primers to retrieve unknown sequence adjacent to a known locus. First, genomic DNA is amplified in the presence of primer to a linker sequence and a primer specific to the known region. The amplified sequences are subjected to a second round of PCR with the same linker primer and another specific primer internal to the first one. Products of each round of PCR are transcribed with an appropriate RNA polymerase and sequenced using reverse transcriptase.

Inverse PCR can be used to amplify or extend sequences using divergent primers based on a known region (Triglia T et al (1988) Nucleic Acids Res 16:8186). The primers may be designed using OLIGO 4.06 Primer Analysis Software (1992; National Biosciences Inc, Plymouth Minn.), or another appropriate program, to be 22-30 nucleotides in length, to have a GC content of 50% or more, and to anneal to the target sequence at temperatures about 68°-72° C. The method uses several restriction enzymes to generate a suitable fragment in the known region of a gene. The fragment is then circularized by intramolecular ligation and used as a PCR template.

Capture PCR (Lagerstrom M et al (1991) PCR Methods Applic 1:111-119) is a method for PCR amplification of DNA fragments adjacent to a known sequence in human and yeast artificial chromosome DNA. Capture PCR also requires multiple restriction enzyme digestions and ligations to place an engineered double-stranded sequence into an unknown portion of the DNA molecule before PCR.

Parker J D et al (1991; Nucleic Acids Res 19:3055-3060), teach walking PCR, a method for targeted gene walking which permits retrieval of unknown sequence. PROMOTERFINDER, a new kit available from Clontech (Palo Alto Calif.) uses PCR, nested primers and PromoterFinder libraries to walk in genomic DNA. This process avoids the need to screen libraries and is useful in finding intron/exon junctions.

Preferred libraries for screening for full length cDNAs are ones that have been size-selected to include larger cDNAs. Also, random primed libraries are preferred in that they will contain more sequences which contain the 5′ and upstream regions of genes. A randomly primed library may be particularly useful if an oligo d(T) library does not yield a full-length cDNA. Genomic libraries are useful for extension into the 5′ nontranslated regulatory region.

A new method for analyzing either the size or confirming the nucleotide sequence of sequencing or PCR products is capillary electrophoresis. Systems for rapid sequencing are available from Perkin Elmer, Beckman Instruments (Fullerton Calif.), and other companies. Capillary sequencing employs flowable polymers for electrophoretic separation, four different fluorescent dyes (one for each nucleotide) which are laser activated, and detection of the emitted wavelengths by a charge coupled devise camera. Output/light intensity is converted to electrical signal using appropriate software (eg. GENOTYPER and SEQUENCE NAVIGATOR from Perkin Elmer) and the entire process from loading of samples to computer analysis and electronic data display is computer controlled. Capillary electrophoresis is particularly suited to the sequencing of small pieces of DNA which might be present in limited amounts in a particular sample. The reproducible sequencing of up to 350 bp of M13 phage DNA in 30 min has been reported (Ruiz-Martinez M C et al (1993) Anal Chem 65:2851-2858).

### Expression of the Nucleotide Sequence

In accordance with the present invention, polynucleotide sequences which encode MCPP, fragments of the polypeptide, fusion proteins or functional equivalents thereof may be used in recombinant DNA molecules that direct the expression of MCPP in appropriate host cells. Due to the inherent degeneracy of the genetic code, other DNA sequences which encode substantially the same or a functionally equivalent amino acid sequence, may be used to clone and express MCPP. As will be understood by those of skill in the art, it may be advantageous to produce MCPP-encoding nucleotide sequences possessing non-naturally occurring codons. Codons preferred by a particular prokaryotic or eukaryotic host (Murray E et al (1989) Nucleic Acids Res 17:477-508) can be selected, for example, to increase the rate of MCPP expression or to produce recombinant RNA transcripts having desirable properties, such as a longer half-life, than transcripts produced from naturally occurring sequence.

The nucleotide sequences of the present invention can be engineered in order to alter a MCPP coding sequence for a variety of reasons, including but not limited to, alterations which modify the cloning, processing and/or expression of the gene product. For example, mutations may be introduced using techniques which are well known in the art, eg, site-directed mutagenesis to insert new restriction sites, to alter glycosylation patterns, to change codon preference, to produce splice variants, etc.

In another embodiment of the invention, a natural, modified or recombinant MCPP sequence may be ligated to a heterologous sequence to encode a fusion protein. For example, for screening of peptide libraries for inhibitors of MCPP activity, it may be useful to encode a chimeric MCPP protein that is recognized by a commercially available antibody. A fusion protein may also be engineered to contain a cleavage site located between a MCPP sequence and the heterologous protein sequence, so that the MCPP may be cleaved and substantially purified away from the heterologous moiety.

In an alternate embodiment of the invention, the coding sequence of MCPP could be synthesized, whole or in part, using chemical methods well known in the art (see Caruthers M H et al (1980) Nucleic Acids Symp Ser 7:215-223, Horn T et al(1980) Nucleic Acids Symp Ser 7:225-232, etc). Alternatively, the protein itself could be produced using chemical methods to synthesize a MCPP amino acid sequence, whole or in part. For example, peptide synthesis can be performed using various solid-phase techniques (Roberge J Y et al (1995) Science 269:202-204) and automated synthesis may be achieved, for example, using the ABI 431A Peptide Synthesizer (Perkin Elmer) in accordance with the instructions provided by the manufacturer.

The newly synthesized peptide can be substantially purified by preparative high performance liquid chromatography (eg, Creighton (1983) Proteins, Structures and Molecular Principles, W H Freeman and Co, New York N.Y.). The composition of the synthetic peptides may be confirmed by amino acid analysis or sequencing (eg, the Edman degradation procedure; Creighton, supra). Additionally the amino acid sequence of MCPP, or any part thereof, may be altered during direct synthesis and/or combined using chemical methods with sequences from other proteins, or any part thereof, to produce a variant polypeptide.

### Expression Systems

In order to express a biologically active MCPP, the nucleotide sequence encoding MCPP or its functional equivalent, is inserted into an appropriate expression vector, ie, a vector which contains the necessary elements for the transcription and translation of the inserted coding sequence.

Methods which are well known to those skilled in the art can be used to construct expression vectors containing a MCPP coding sequence and appropriate transcriptional or translational controls. These methods include in vitro recombinant DNA techniques, synthetic techniques and in vivo recombination or genetic recombination. Such techniques are described in Sambrook et al (1989) Molecular Cloning, A Laboratory Manual, Cold Spring Harbor Press, Plainview N.Y. and Ausubel F M et al (1989) Current Protocols in Molecular Biology, John Wiley & Sons, New York N.Y.

A variety of expression vector/host systems may be utilized to contain and express a MCPP coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (eg, baculovirus); plant cell systems transfected with virus expression vectors (eg, cauliflower mosaic virus, CaMV; tobacco mosaic virus, TMV) or transformed with bacterial expression vectors (eg, Ti or pBR322 plasmid); or animal cell systems.

The “control elements” or “regulatory sequences” of these systems vary in their strength and specificities and are those nontranslated regions of the vector, enhancers, promoters, and 3′ untranslated regions, which interact with host cellular proteins to carry out transcription and translation. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, LaJolla Calif.) or PSPORT1 (Gibco BRL) and ptrp-lac hybrids and the like may be used. The baculovirus polyhedrin promoter may be used in insect cells. Promoters or enhancers derived from the genomes of plant cells (eg, heat shock, RUBISCO; and storage protein genes) or from plant viruses (eg, viral promoters or leader sequences) may be cloned into the vector. In mammalian cell systems, promoters from the mammalian genes or from mammalian viruses are most appropriate. If it is necessary to generate a cell line that contains multiple copies of MCPP, vectors based on SV40 or EBV may be used with an appropriate selectable marker.

In bacterial systems, a number of expression vectors may be selected depending upon the use intended for MCPP. For example, when large quantities of MCPP are needed for the induction of antibodies, vectors which direct high level expression of fusion proteins that are readily purified may be desirable. Such vectors include, but are not limited to, the multifunctional E. coli cloning and expression vectors such as BLUESCRIPT (Stratagene), in which the MCPP coding sequence may be ligated into the vector in frame with sequences for the amino-terminal Met and the subsequent 7 residues of β-galactosidase so that a hybrid protein is produced; pIN vectors (Van Heeke & Schuster (1989) J Biol Chem 264:5503-5509); and the like. PGEX vectors (Promega, Madison Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems are designed to include heparin, thrombin or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase and PGH may be used. For reviews, see Ausubel et al (supra) and Bitter G A et al (1987) Methods Enzymol 153:516-544.

In cases where plant expression vectors are used, the expression of a sequence encoding MCPP may be driven by any of a number of promoters. For example, viral promoters such as the 35S and 19S promoters of CamV (Brisson et al (1984) Nature 310:511-514) may be used alone or in combination with the omega leader sequence from TMV (Takamatsu et al (1987) EMBO J 6:307-311). Alternatively, plant promoters such as the small subunit of RUBISCO (Coruzzi et al (1984) EMBO J 3:1671-1680; Broglie et al (1984) Science 224:838-843); or heat shock promoters (Winter and Sinibaldi R M (1991) Results Probl Cell Differ 17:85-105) may be used. These constructs can be introduced into plant cells by direct DNA transformation or pathogen-mediated transfection. For reviews of such techniques, see Hobbs S or Murry L E in McGraw Hill Yearbook of Science and Technology (1992) McGraw Hill New York N.Y., pp 191-196 or Weissbach and Weissbach (1988) Methods for Plant Molecular Biology, Academic Press, New York N.Y., pp 421-463.

An alternative expression system which could be used to express MCPP is an insect system. In one such system, Autographa californica nuclear polyhedrosis virus (AcNPV) is used as a vector to express foreign genes in Spodoptera frugiperda cells or in Trichoplusia larvae. The MCPP coding sequence may be cloned into a nonessential region of the virus, such as the polyhedrin gene, and placed under control of the polyhedrin promoter. Successful insertion of MCPP will render the polyhedrin gene inactive and produce recombinant virus lacking coat protein coat. The recombinant viruses are then used to infect S. frugiperda cells or Trichoplusia larvae in which MCPP is expressed (Smith et al (1983) J Virol 46:584; Engelhard E K et al (1994) Proc Natl Acad Sci USA 91:3224-3227).

In mammalian host cells, a number of viral-based expression systems may be utilized. In cases where an adenovirus is used as an expression vector, a MCPP coding sequence may be ligated into an adenovirus transcription/translation complex consisting of the late promoter and tripartite leader sequence. Insertion in a nonessential E1 or E3 region of the viral genome will result in a viable virus capable of expressing MCPP in infected host cells (Logan and Shenk (1984) Proc Natl Acad Sci USA 81:3655-3659). In addition, transcription enhancers, such as the rous sarcoma virus (RSV) enhancer, may be used to increase expression in mammalian host cells.

Specific initiation signals may also be required for efficient translation of a MCPP sequence. These signals include the ATG initiation codon and adjacent sequences. In cases where MCPP, its initiation codon and upstream sequences are inserted into the appropriate expression vector, no additional translational control signals may be needed. However, in cases where only coding sequence, or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression may be enhanced by the inclusion of enhancers appropriate to the cell system in use (Scharf D et al (1994) Results Probl Cell Differ 20:125-162; Bittner et al (1987) Methods Enzymol 153:516-544).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, etc have specific cellular machinery and characteristic mechanisms for such post-translational activities and may be chosen to ensure the correct modification and processing of the introduced, foreign protein.

For long-term, high-yield production of recombinant proteins, stable expression is preferred. For example, cell lines which stably express MCPP may be transformed using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clumps of stably transformed cells can be proliferated using tissue culture techniques appropriate to the cell type.

Any number of selection systems may be used to recover transformed cell lines. These include, but are not limited to, the herpes simplex virus thymidine kinase (Wigler M et al (1977) Cell 11:223-232) and adenine phosphoribosyltransferase (Lowy I et al (1980) Cell 22:817-823) genes which can be employed in tk- or aprt- cells, respectively. Also, antimetabolite, antibiotic or herbicide resistance can be used as the basis for selection; for example, dhfr which confers resistance to methotrexate (Wigler M et al (1980) Proc Natl Acad Sci USA 77:3567-3570); npt, which confers resistance to the aminoglycosides neomycin and G-418 (Colbere-Garapin F et al (1981) J Mol Biol 150:1-14) and als and pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman S C and R C Mulligan (1988) Proc Natl Acad Sci USA 85:8047-8051). Recently, the use of visible markers has gained popularity with such markers as anthocyanins, β glucuronidase and its substrate, GUS, and luciferase and its substrate, luciferin, being widely used not only to identify transformants, but also to quantify the amount of transient or stable protein expression attributable to a specific vector system (Rhodes C A et al (1995) Methods Mol Biol 55:121-131).

### Identification of Transformants Containing the Polynucleotide Sequence

Although the presence/absence of marker gene expression suggests that the gene of interest is also present, its presence and expression should be confirmed. For example, if the MCPP is inserted within a marker gene sequence, recombinant cells containing MCPP can be identified by the absence of marker gene function. Alternatively, a marker gene can be placed in tandem with a MCPP sequence under the control of a single promoter. Expression of the marker gene in response to induction or selection usually indicates expression of the tandem MCPP as well.

Alternatively, host cells which contain the coding sequence for MCPP and express MCPP may be identified by a variety of procedures known to those of skill in the art. These procedures include, but are not limited to, DNA-DNA or DNA-RNA hybridization and protein bioassay or immunoassay techniques which include membrane, solution, or chip based technologies for the detection and/or quantification of the nucleic acid or protein.

The presence of the MCPP polynucleotide sequence can be detected by DNA-DNA or DNA-RNA hybridization or amplification using probes, portions or fragments of MCPP. Nucleic acid amplification based assays involve the use of oligonucleotides or oligomers based on the MCPP sequence to detect transformants containing MCPP DNA or RNA. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides which can be used as a probe or amplimer.

A variety of protocols for detecting and measuring the expression of MCPP, using either polyclonal or monoclonal antibodies specific for the protein are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes on MCPP is preferred, but a competitive binding assay may be employed. These and other assays are described, among other places, in Hampton R et al (1990, Serological Methods, a Laboratory Manual, APS Press, St Paul Minn.) and Maddox D E et al (1983, J Exp Med 158:1211).

A wide variety of labels and conjugation techniques are known by those skilled in the art and can be used in various nucleic acid and amino acid assays. Means for producing labeled hybridization or PCR probes for detecting sequences related to MCPP include oligolabeling, nick translation, end-labeling or PCR amplification using a labeled nucleotide. Alternatively, the MCPP sequence, or any portion of it, may be cloned into a vector for the production of an mRNA probe. Such vectors are known in the art, are commercially available, and may be used to synthesize RNA probes in vitro by addition of an appropriate RNA polymerase such as T7, T3 or SP6 and labeled nucleotides.

A number of companies such as Pharmacia Biotech (Piscataway N.J.), Promega (Madison Wis.), and US Biochemical Corp (Cleveland Ohio) supply commercial kits and protocols for these procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. Nos. 3,817,837; 3,850,752; 3,939,350; 3,996,345; 4,277,437; 4,275,149 and 4,366,241. Also, recombinant immunoglobulins may be produced as shown in U.S. Pat. No. 4,816,567 incorporated herein by reference.

### Purification of MCPP

Host cells transformed with a MCPP nucleotide sequence may be cultured under conditions suitable for the expression and recovery of the encoded protein from cell culture. The protein produced by a recombinant cell may be secreted or contained intracellularly depending on the sequence and/or the vector used. As will be understood by those of skill in the art, expression vectors containing MCPP can be designed with signal sequences which direct secretion of MCPP through a prokaryotic or eukaryotic cell membrane. Other recombinant constructions may join MCPP to nucleotide sequence encoding a polypeptide domain which will facilitate purification of soluble proteins (Kroll D J et al (1993) DNA Cell Biol 12:441-453; cf discussion of vectors infra containing fusion proteins).

MCPP may also be expressed as a recombinant protein with one or more additional polypeptide domains added to facilitate protein purification. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor XA or enterokinase (Invitrogen, San Diego Calif.) between the purification domain and MCPP is useful to facilitate purification. One such expression vector provides for expression of a fusion protein compromising an MCPP and contains nucleic acid encoding 6 histidine residues followed by thioredoxin and an enterokinase cleavage site. The histidine residues facilitate purification on IMIAC (immobilized metal ion affinity chromatography as described in Porath et al (1992) Protein Expression and Purification 3:263-281) while the enterokinase cleavage site provides a means for purifying the chemokine from the fusion protein.

In addition to recombinant production, fragments of MCPP may be produced by direct peptide synthesis using solid-phase techniques (c f Stewart et al (1969) Solid-Phase Peptide Synthesis, W H Freeman Co, San Francisco; Merrifield J (1963) J Am Chem Soc 85:2149-2154). In vitro protein synthesis may be performed using manual techniques or by automation. Automated synthesis may be achieved, for example, using Applied Biosystems 431A Peptide Synthesizer (Perkin Elmer, Foster City Calif.) in accordance with the instructions provided by the manufacturer. Various fragments of MCPP may be chemically synthesized separately and combined using chemical methods to produce the full length molecule.

### Uses of MCPP

The rationale for use of the polypeptide sequences disclosed herein is based on the chemical and structural homology among the novel human MCPP and related CC chemokines, hJE-2/MCP-2 (GI 338809) and MCP-3 (GI 288397). Expression of the genes in this family is often cell or tissue specific and associated with autoimmune, cancerous, inflammatory, infectious, or hereditary conditions. These molecules and their variants will be found in many different tissues where they carry out the characteristic activity of a CC chemokines, diapedesis, extravasion or chemotactic functions.

The protein may be used to screen for receptors on the surface of leukocytes, cells of lymphoid or cancerous origin. It may also be used as “bait’ to fish for effective biological or organic pharmaceutical molecules. Similarly obtained antibodies, antagonists and inhibitors of MCPP may be used to modulate the chemoattraction of monocytes and macrophages to tissues such as asthmatic lung and noninsulin dependent diabetic pancreas where excessive MCPP expression can cause tissue damage. Regulation of the amount of MCPP is also important in diseases such as AIDS and rheumatoid arthritis.

### MCPP Antibodies

MCPP-specific antibodies are useful for the diagnosis of conditions and diseases associated with expression of MCPP. MCPP for antibody induction does not require biological activity; however, the protein fragment, or oligopeptide must be antigenic. Peptides used to induce specific antibodies may have an amino acid sequence consisting of at least five amino acids, preferably at least 10 amino acids. They should mimic a portion of the amino acid sequence of the natural protein and may contain the entire amino acid sequence of a small, naturally occurring molecule. Short stretches of MCPP amino acids may be fused with those of another protein such as keyhole limpet hemocyanin and antibody produced against the chimeric molecule. Procedures well known in the art can be used for the production of antibodies to MCPP. Such antibodies include, but are not limited to, polyclonal, monoclonal, chimeric, single chain, Fab fragments and fragments produced by a Fab expression library. Neutralizing antibodies, ie, those which inhibit dimer formation, are especially preferred for diagnostics and therapeutics.

For the production of antibodies, various hosts including goats, rabbits, rats, mice, etc may be immunized by injection with MCPP or any portion, fragment or oligopeptide which retains antigenic properties. Depending on the host species, various adjuvants may be used to increase immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

Monoclonal antibodies to MCPP may be prepared using any technique which provides for the production of antibody molecules by continuous cell lines in culture. These include but are not limited to the hybridoma technique originally described by Koehler and Milstein (1975 Nature 256:495-497), the human B-cell hybridoma technique (Kosbor et al (1983) Immunol Today 4:72; Cote et al (1983) Proc Natl Acad Sci USA 80:2026-2030) and the EBV-hybridoma technique (Cole et al (1985) Monoclonal Antibodies and Cancer Therapy, Alan R Liss Inc, New York N.Y., pp 77-96).

In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci USA 81:6851-6855; Neuberger et al (1984) Nature 312:604-608; Takeda et al (1985) Nature 314:452-454). Alternatively, techniques described for the production of single chain antibodies (U.S. Pat. No. 4,946,778) can be adapted to produce MCPP-specific single chain antibodies.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci USA 86:3833-3837), and Winter G and Milstein C (1991; Nature 349:293-299).

Antibody fragments which contain specific binding sites for MCPP may also be generated. For example, such fragments include, but are not limited to, the F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed to allow rapid and easy identification of monoclonal Fab fragments with the desired specificity (Huse W D et al (1989) Science 256:1275-1281).

A variety of protocols for competitive binding or immunoradiometric assays using either polyclonal or monoclonal antibodies with established specificities are well known in the art. Such immunoassays typically involve the formation of complexes between MCPP and its specific antibody and the measurement of complex formation. A two-site, monoclonal-based immunoassay utilizing monoclonal antibodies reactive to two noninterfering epitopes on a specific MCPP protein is preferred, but a competitive binding assay may also be employed. These assays are described in Maddox D E et al (1983, J Exp Med 158:1211).

### Diagnostic Assays Using MCPP Specific Antibodies

Particular MCPP antibodies are useful for the diagnosis of conditions or diseases characterized by expression of MCPP or in assays to monitor patients being treated with MCPP, agonists or inhibitors. Diagnostic assays for MCPP include methods utilizing the antibody and a label to detect MCPP in human body fluids or extracts of cells or tissues. The polypeptides and antibodies of the present invention may be used with or without modification. Frequently, the polypeptides and antibodies will be labeled by joining them, either covalently or noncovalently, with a reporter molecule. A wide variety of reporter molecules are known, several of which were described above.

A variety of protocols for measuring MCPP, using either polyclonal or monoclonal antibodies specific for the respective protein are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes on MCPP is preferred, but a competitive binding assay may be employed. These assays are described, among other places, in Maddox, D E et al (1983, J Exp Med 158:1211).

In order to provide a basis for diagnosis, normal or standard values for MCPP expression must be established. This is accomplished by combining body fluids or cell extracts taken from normal subjects, either animal or human, with antibody to MCPP under conditions suitable for complex formation which are well known in the art. The amount of standard complex formation may be quantified by comparing various artificial membranes containing known quantities of MCPP with both control and disease samples from biopsied tissues. Then, standard values obtained from normal samples may be compared with values obtained from samples from subjects potentially affected by disease. Deviation between standard and subject values establishes the presence of disease state.

### Drug Screening

MCPP or its catalytic or antigenic fragments can be used for screening therapeutic compounds in any of a variety of drug screening techniques. The fragment employed in such a test may be free in solution, affixed to a solid support, borne on a cell surface, or located intracellularly. The formation of binding complexes, between MCPP and the agent being tested, may be measured.

Another technique for drug screening which provides for high throughput screening of compounds having suitable binding affinity to the MCPP is described in detail in “Determination of Amino Acid Sequence Antigenicity” by Geysen H N, WO Application 84/03564, published on Sep. 13, 1984, and incorporated herein by reference. In summary, large numbers of different small peptide test compounds are synthesized on a solid substrate, such as plastic pins or some other surface. The peptide test compounds are reacted with fragments of MCPP and washed. Bound MCPP is then detected by methods well known in the art. Substantially purified MCPP can also be coated directly onto plates for use in the aforementioned drug screening techniques. Alternatively, non-neutralizing antibodies can be used to capture the peptide and immobilize it on a solid support.

This invention also contemplates the use of competitive drug screening assays in which neutralizing antibodies capable of binding MCPP specifically compete with a test compound for binding MCPP. In this manner, the antibodies can be used to detect the presence of any peptide which shares one or more antigenic determinants with MCPP.

### Uses of the Polynucleotide Encoding MCPP

A polynucleotide encoding MCPP, or any part thereof, may be used for diagnostic and/or therapeutic purposes. For diagnostic purposes, the MCPP of this invention may be used to detect and quantitate gene expression in body fluids or biopsied tissues in which expression of MCPP may be implicated. The diagnostic assay is useful to distinguish between absence, presence, and excess expression of MCPP and to monitor regulation of MCPP levels during therapeutic intervention. Included in the scope of the invention are oligonucleotide sequences, antisense RNA and DNA molecules, and PNAs.

Conditions, disorders or diseases in which MCPP activity may be implicated specifically include rheumatoid arthritis, noninsulin dependent diabetes mellitus (NIDDM) and cancers, such as cancers of the bladder and breast as shown in FIG. 5, but they may also include conditions such as viral, bacterial, fungal or helminthic infections; allergic or asthmatic responses; mechanical injury associated with trauma; arteriosclerosis, atherogenesis or collagen vascular diseases; leukemia, lymphomas or carcinomas; or other conditions which involve monocytes or macrophages.

Another aspect of the subject invention is to provide for hybridization or PCR probes which are capable of detecting polynucleotide sequences, including genomic sequences, encoding MCPP or closely related molecules. The specificity of the probe, whether it is made from a highly conserved region, eg, 10 unique nucleotides in the 5′ regulatory region, or a less conserved region, eg, 20 nucleotides downstream from the definitive CC motif, and the stringency of the hybridization or amplification (high, intermediate or low) will determine whether the probe identifies only native MCPP, allelles, or related chemokine molecules.

Hybridization probes used for the detection of related sequences should preferably contain at least 50% of the nucleotides from MCPP encoding sequences. The hybridization probes of the subject invention may be derived from the nucleotide sequence of SEQ ID NO:2 or from genomic sequence including promoter, enhancer elements and introns of the naturally occurring MCPP. Hybridization probes may be labeled by a variety of reporter groups, including radionuclides such as 32P or 35S, or enzymatic labels such as alkaline phosphatase coupled to the probe via avidin/biotin coupling systems, and the like.

Other means for producing specific hybridization probes for MCPP DNAs include the cloning of nucleic acid sequences encoding MCPP or MCPP derivatives into vectors for the production of mRNA probes. Such vectors are known in the art and are commercially available and may be used to synthesize RNA probes in vitro by means of the addition of the appropriate RNA polymerase as T7 or SP6 RNA polymerase and the appropriate radioactively labeled nucleotides.

### Diagnostics

Polynucleotide sequences encoding MCPP may be used for the diagnosis of conditions or diseases with which the expression of MCPP is associated. For example, polynucleotide sequences encoding MCPP may be used in hybridization or PCR assays of fluids or tissues from biopsies to detect MCPP expression. The form of such qualitative or quantitative methods may include Southern or northern analysis, dot blot or other membrane-based technologies; PCR technologies; dip stick, pin, chip and ELISA technologies. All of these techniques are well known in the art and are the basis of many commercially available diagnostic kits.

The MCPP nucleotide sequences disclosed herein provide the basis for assays that detect activation or induction associated with inflammation or disease. The MCPP nucleotide sequence may be labeled by methods known in the art and added to a fluid or tissue sample from a patient under conditions suitable for the formation of hybridization complexes. After an incubation period, the sample is washed with a compatible fluid which optionally contains a dye (or other label requiring a developer) if the nucleotide has been labeled with an enzyme. After the compatible fluid is rinsed off, the dye is quantitated and compared with a standard. If the amount of dye in the biopsied or extracted sample is significantly elevated over that of a comparable control sample, the nucleotide sequence has hybridized with nucleotide sequences in the sample, and the presence of elevated levels of MCPP nucleotide sequences in the sample indicates the presence of the associated inflammation and/or disease.

Such assays may also be used to evaluate the efficacy of a particular therapeutic treatment regime in animal studies, in clinical trials, or in monitoring the treatment of an individual patient. In order to provide a basis for the diagnosis of disease, a normal or standard profile for MCPP expression must be established. This is accomplished by combining body fluids or cell extracts taken from normal subjects, either animal or human, with MCPP, or a portion thereof, under conditions suitable for hybridization or amplification. Standard hybridization may be quantified by comparing the values obtained for normal subjects with a dilution series of MCPP run in the same experiment where a known amount of substantially purified MCPP is used. Standard values obtained from normal samples may be compared with values obtained from samples from patients affected by MCPP-associated diseases. Deviation between standard and subject values establishes the presence of disease.

Once disease is established, a therapeutic agent is administered and a treatment profile is generated. Such assays may be repeated on a regular basis to evaluate whether the values in the profile progress toward or return to the normal or standard pattern. Successive treatment profiles may be used to show the efficacy of treatment over a period of several days or several months.

PCR as described in U.S. Pat. Nos. 4,683,195 and 4,965,188 provides additional uses for oligonucleotides based upon the MCPP sequence. Such oligomers are generally chemically synthesized, but they may be generated enzymatically or produced from a recombinant source. Oligomers generally comprise two nucleotide sequences, one with sense orientation (5′->3′) and one with antisense (3′<-5′), employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Additionally, methods to quantitate the expression of a particular molecule include radiolabeling (Melby P C et al 1993 J Immunol Methods 159:235-244) or biotinylating (Duplaa C et al 1993 Anal Biochem 212:229-236) nucleotides, coamplification of a control nucleic acid, and standard curves onto which the experimental results are interpolated. Quantitation of multiple samples may be speeded up by running the assay in an ELISA format where the oligomer of interest is presented in various dilutions and a spectrophotometric or colorimetric response gives rapid quantitation. A definitive diagnosis of this type may allow health professionals to begin aggressive treatment and prevent further worsening of the condition. Similarly, further assays can be used to monitor the progress of a patient during treatment. Furthermore, the nucleotide sequences disclosed herein may be used in molecular biology techniques that have not yet been developed, provided the new techniques rely on properties of nucleotide sequences that are currently known such as the triplet genetic code, specific base pair interactions, and the like.

### Therapeutics

Based upon its homology to other CC chemokines, particularly MCP-1, and MCP-3, and its expression profile, the MCPP polynucleotide disclosed herein may be useful in the treatment of disorders such as asthma, rheumatoid arthritis, NIDDM or cancer of the breast or bladder.

Expression vectors derived from retroviruses, adenovirus, herpes or vaccinia viruses, or from various bacterial plasmids, may be used for delivery of nucleotide sequences to the targeted organ, tissue or cell population. Methods which are well known to those skilled in the art can be used to construct recombinant vectors which will express antisense MCPP. See, for example, the techniques described in Sambrook et al (supra) and Ausubel et al (supra).

The polynucleotides comprising full length cDNA sequence and/or its regulatory elements enable researchers to use MCPP as an investigative tool in sense (Youssoufian H and H F Lodish 1993 Mol Cell Biol 13:98-104) or antisense (Eguchi et al (1991) Annu Rev Biochem 60:631-652) regulation of gene function. Such technology is now well known in the art, and sense or antisense oligomers, or larger fragments, can be designed from various locations along the coding or control regions.

Genes encoding MCPP can be turned off by transfecting a cell or tissue with expression vectors which express high levels of a desired MCPP fragment. Such constructs can flood cells with untranslatable sense or antisense sequences. Even in the absence of integration into the DNA, such vectors may continue to transcribe RNA molecules until all copies are disabled by endogenous nucleases. Transient expression may last for a month or more with a non-replicating vector (Mettler I, personal communication) and even longer if appropriate replication elements are part of the vector system.

As mentioned above, modifications of gene expression can be obtained by designing antisense molecules, DNA, RNA or PNA, to the control regions of MCPP, ie, the promoters, enhancers, and introns. Oligonucleotides derived from the transcription initiation site, eg, between −10 and +10 regions of the leader sequence, are preferred. The antisense molecules may also be designed to block translation of mRNA by preventing the transcript from binding to ribosomes. Similarly, inhibition can be achieved using “triple helix” base-pairing methodology. Triple helix pairing compromises the ability of the double helix to open sufficiently for the binding of polymerases, transcription factors, or regulatory molecules. Recent therapeutic advances using triplex DNA were reviewed by Gee J E et al (In: Huber B E and B I Carr (1994) Molecular and Immunologic Approaches, Futura Publishing Co, Mt Kisco N.Y.).

Ribozymes are enzymatic RNA molecules capable of catalyzing the specific cleavage of RNA. The mechanism of ribozyme action involves sequence-specific hybridization of the ribozyme molecule to complementary target RNA, followed by endonucleolytic cleavage. Within the scope of the invention are engineered hammerhead motif ribozyme molecules that can specifically and efficiently catalyze endonucleolytic cleavage of MCPP.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Antisense molecules and ribozymes of the invention may be prepared by any method known in the art for the synthesis of RNA molecules. These include techniques for chemically synthesizing oligonucleotides such as solid phase phosphoramidite chemical synthesis. Alternatively, RNA molecules may be generated by in vitro and in vivo transcription of DNA sequences encoding MCPP. Such DNA sequences may be incorporated into a wide variety of vectors with suitable RNA polymerase promoters such as T7 or SP6. Alternatively, antisense cDNA constructs that synthesize antisense RNA constitutively or inducibly can be introduced into cell lines, cells or tissues.

RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine and wybutosine as well as acetyl-, methyl-, thio- and similarly modified forms of adenine, cytidine, guanine, thymine, and uridine which are not as easily recognized by endogenous endonucleases.

Methods for introducing vectors into cells or tissues include those methods discussed infra and which are equally suitable for in vivo, in vitro and ex vivo therapy. For ex vivo therapy, vectors are introduced into stem cells taken from the patient and clonally propagated for autologous transplant back into that same patient is presented in U.S. Pat. Nos. 5,399,493 and 5,437,994, disclosed herein by reference. Delivery by transfection and by liposome are quite well known in the art.

Furthermore, the nucleotide sequences for MCPP disclosed herein may be used in molecular biology techniques that have not yet been developed, provided the new techniques rely on properties of nucleotide sequences that are currently known, including but not limited to such properties as the triplet genetic code and specific base pair interactions.

### Detection and Mapping of Related Polynucleotide Sequences

The nucleic acid sequence for MCPP can also be used to generate hybridization probes for mapping the naturally occurring genomic sequence. The sequence may be mapped to a particular chromosome or to a specific region of the chromosome using well known techniques. These include in situ hybridization to chromosomal spreads, flow-sorted chromosomal preparations, or artificial chromosome constructions such as yeast artificial chromosomes, bacterial artificial chromosomes, bacterial P1 constructions or single chromosome cDNA libraries as reviewed in Price C M (1993; Blood Rev 7:127-34) and Trask B J (1991; Trends Genet 7:149-154).

The technique of fluorescent in situ hybridization of chromosome spreads has been described, among other places, in Verma et al (1988) Human Chromosomes: A Manual of Basic Techniques, Pergamon Press, New York N.Y. Fluorescent in situ hybridization of chromosomal preparations and other physical chromosome mapping techniques may be correlated with additional genetic map data. Examples of genetic map data can be found in the 1994 Genome Issue of Science (265:1981f). Correlation between the location of a MCPP on a physical chromosomal map and a specific disease (or predisposition to a specific disease) may help delimit the region of DNA associated with that genetic disease. The nucleotide sequences of the subject invention may be used to detect differences in gene sequences between normal, carrier or affected individuals.

In situ hybridization of chromosomal preparations and physical mapping techniques such as linkage analysis using established chromosomal markers are invaluable in extending genetic maps. A recent example of an STS based map of the human genome was recently published by the Whitehead-MIT Center for Genomic Research (Hudson T J et al (1995) Science 270:1945-1954). Often the placement of a gene on the chromosome of another mammalian species such as mouse (Whitehead Institute/MIT Center for Genome Research, Genetic Map of the Mouse, Database Release 10, Apr. 28, 1995) may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once a disease or syndrome, such as ataxia telangiectasia (AT), has been crudely localized by genetic linkage to a particular genomic region, for example, AT to 11q22-23 (Gatti et al (1988) Nature 336:577-580), any sequences mapping to that area may represent associated or regulatory genes for further investigation. The nucleotide sequence of the subject invention may also be used to detect differences in the chromosomal location due to translocation, inversion, etc. among normal, carrier or affected individuals.

### Pharmaceutical Compositions

The present invention relates to pharmaceutical compositions which may comprise nucleotides, proteins, antibodies, agonists, antagonists, or inhibitors, alone or in combination with at least one other agent, such as stabilizing compound, which may be administered in any sterile, biocompatible pharmaceutical carrier, including, but not limited to, saline, buffered saline, dextrose, and water. Any of these molecules can be administered to a patient alone, or in combination with other agents, drugs or hormones, in pharmaceutical compositions where it is mixed with excipient(s) or pharmaceutically acceptable carriers. In one embodiment of the present invention, the pharmaceutically acceptable carrier is pharmaceutically inert.

### Administration of Pharmaceutical Compositions

Administration of pharmaceutical compositions is accomplished orally or parenterally. Methods of parenteral delivery include topical, intra-arterial (directly to the tumor), intramuscular, subcutaneous, intramedullary, intrathecal, intraventricular, intravenous, intraperitoneal, or intranasal administration. In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of “Remington's Pharmaceutical Sciences” (Maack Publishing Co, Easton Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, ie, dosage.

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hanks's solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

### Manufacture and Storage

The pharmaceutical compositions of the present invention may be manufactured in a manner that known in the art, eg, by means of conventional mixing, dissolving, granulating, dragee-making, levigating, emulsifying, encapsulating, entrapping or lyophilizing processes.

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

After pharmaceutical compositions comprising a compound of the invention formulated in a acceptable carrier have been prepared, they can be placed in an appropriate container and labeled for treatment of an indicated condition. For administration of MCPP, such labeling would include amount, frequency and method of administration.

### Therapeutically Effective Dose

Pharmaceutical compositions suitable for use in the present invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, eg, of neoplastic cells, or in animal models, usually mice, rabbits, dogs, or pigs. The animal model is also used to achieve a desirable concentration range and route of administration. Such information can then be used to determine useful doses and routes for administration in humans.

A therapeutically effective dose refers to that amount of protein or its antibodies, antagonists, or inhibitors which ameliorate the symptoms or condition. Therapeutic efficacy and toxicity of such compounds can be determined by standard pharmaceutical procedures in cell cultures or experimental animals, eg, ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors which may be taken into account include the severity of the disease state, eg, tumor size and location; age, weight and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature. See U.S. Pat. Nos. 4,657,760; 5,206,344; or 5,225,212. Those skilled in the art will employ different formulations for nucleotides than for proteins or their inhibitors. Similarly, delivery of polynucleotides or polypeptides will be specific to particular cells, conditions, locations, etc.

It is contemplated, for example, that an antagonist or inhibitor of MCPP can be delivered to the lungs in a suitable formulation as a therapeutic agent. Such delivery would prevent inappropriate activation of monocytes and macrophages in allergies, asthma or emphysema.

The examples below are provided to illustrate the subject invention and are not included for the purpose of limiting the invention.

### EXAMPLES

### I CDNA Library Construction

The nontumorous breast (BRSTNOT05) cDNA library was constructed from the breast tissue of a 58 year old Caucasian female. The nontumorous breast tissue was adjacent to tumorous tissue in a patient diagnosed with multicentric invasive grade 4 lobular carcinoma (specimens #0116A and #0116B; Mayo Clinic, Rochester Minn.).

The frozen tissue was homogenized and lysed using a Brinkmann Homogenizer Polytron PT-3000 (Brinkmann Instruments, Westbury N.J.) in guanidinium isothiocyanate solution. The lysate was centrifuged over a 5.7 M CsCl cushion using an Beckman SW28 rotor in a Beckman L8-70M Ultracentrifuge (Beckman Instruments) for 18 hours at 25,000 rpm at ambient temperature. The RNA was extracted with acid phenol pH 4.0, precipitated using 0.3 M sodium acetate and 2.5 volumes of ethanol, resuspended in RNAse-free water and DNase treated at 37° C. The RNA extraction was repeated with acid phenol chloroform pH 8.0 and precipitated with sodium acetate and ethanol as before. The mRNA was then isolated using the QIAGEN OLIGOTEX kit (QIAGEN Inc; Chatsworth Calif.) and used to construct the cDNA library.

The mRNA was handled according to the recommended protocols in the SUPERSCRIPT Plasmid System for cDNA Synthesis and Plasmid Cloning (Cat. #18248-013; Gibco/BRL), cDNAs were fractionated on a Sepharose CL4B column (Cat. #275105-01; Pharmacia), and those cDNAs exceeding 400 bp were ligated into PSPORT1 plasmid. The plasmid PSPORT1 was subsequently transformed into DH5a [symbol] competent cells (Cat. #18258-012; Gibco/BRL).

### II Isolation and Sequencing of CDNA Clones

Plasmid DNA was released from the cells and purified using the REAL PREP 96 Plasmid Kit for Rapid Extraction Alkaline Lysis Plasmid Minipreps (Catalog #26173; QIAGEN, Inc). This kit enables the simultaneous purification of 96 samples in a 96-well block using multi-channel reagent dispensers. The recommended protocol was employed except for the following changes: 1) the bacteria were cultured in 1 ml of sterile Terrific Broth (Catalog #22711, LIFE TECHNOLOGIES) with carbenicillin at 25 mg/L and glycerol at 0.4%; 2) after inoculation, the cultures were incubated for 19 hours and at the end of incubation, the cells were lysed with 0.3 ml of lysis buffer; and 3) following isopropanol precipitation, the plasmid DNA pellet was resuspended in 0.1 ml of distilled water. After the last step in the protocol, samples were transferred to a 96-well block for storage at 4° C.

The cDNAs were sequenced by the method of Sanger F and A R Coulson (1975; J Mol Biol 94:441f), using a Hamilton Micro Lab 2200 (Hamilton, Reno Nev.) in combination with Peltier Thermal Cyclers (PTC200 from MJ Research, Watertown Mass.) and Applied Biosystems 377 DNA Sequencing Systems; and the reading frame was determined.

### III Homology Searching of cDNA Clones and Their Deduced Proteins

Each cDNA was compared to sequences in GenBank using a search algorithm developed by Applied Biosystems and incorporated into the INHERIT 670 Sequence Analysis System. In this algorithm, Pattern Specification Language (TRW Inc, Los Angeles Calif.) was used to determine regions of homology. The three parameters that determine how the sequence comparisons run were window size, window offset, and error tolerance. Using a combination of these three parameters, the DNA database was searched for sequences containing regions of homology to the query sequence, and the appropriate sequences were scored with an initial value. Subsequently, these homologous regions were examined using dot matrix homology plots to distinguish regions of homology from chance matches. Smith-Waterman alignments were used to display the results of the homology search.

Peptide and protein sequence homologies were ascertained using the INHERIT™ 670 Sequence Analysis System in a way similar to that used in DNA sequence homologies. Pattern Specification Language and parameter windows were used to search protein databases for sequences containing regions of homology which were scored with an initial value. Dot-matrix homology plots were examined to distinguish regions of significant homology from chance matches.

BLAST, which stands for Basic Local Alignment Search Tool (Altschul S F (1993) J Mol Evol 36:290-300; Altschul, S F et al (1990) J Mol Biol 215:403-410), was used to search for local sequence alignments. BLAST produces alignments of both nucleotide and amino acid sequences to determine sequence similarity. Because of the local nature of the alignments, BLAST is especially useful in determining exact matches or in identifying homologs. BLAST is useful for matches which do not contain gaps. The fundamental unit of BLAST algorithm output is the High-scoring Segment Pair (HSP).

An HSP consists of two sequence fragments of arbitrary but equal lengths whose alignment is locally maximal and for which the alignment score meets or exceeds a threshold or cutoff score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

### IV Northern Analysis

Northern analysis is a laboratory technique used to detect the presence of a transcript of a gene and involves the hybridization of a labelled nucleotide sequence to a membrane on which RNAs from a particular cell type or tissue have been bound (Sambrook et al. supra).

Analogous computer techniques using BLAST (Altschul S F 1993 and 1990, supra) are used to search for identical or related molecules in nucleotide databases such as GenBank or the LIFESEQ database (Incyte, Palo Alto Calif.). This analysis is much faster than multiple, membrane-based hybridizations. In addition, the sensitivity of the computer search can be modified to determine whether any particular match is categorized as exact or homologous.

The basis of the search is the product score which is defined as:

% sequence identity×% maximum BLAST score

and it takes into acccount both the degree of similarity between two sequences and the length of the sequence match. For example, with a product score of 40, the match is exact within a 1-2% error; and at 70, the match is exact. Homologous molecules are usually identified by selecting those which show product scores between 15 and 40, although lower scores usually identify related molecules.

### V Extension of the Nucleotide Sequence to Recover Regulatory Elements

The nucleic acid sequence encoding MCPP (SEQ ID NO:2) is used to design oligonucleotide primers for obtaining 5′ sequences from genomic libraries. One primer is synthesized to initiate extension in the antisense direction (XLR) and the other is synthesized to extend sequence in the sense direction (XLF). Primers allow the extension of the known MCPP sequence “outward” generating amplicons containing new, unknown nucleotide sequence for the region of interest (U.S. Pat. application No. 08/487,112, filed Jun. 7, 1995, specifically incorporated by reference). The initial primers are designed from the cDNA using OLIGO 4.06 Primer Analysis Software (National Biosciences), or another appropriate program, to be 22-30 nucleotides in length, to have a GC content of 50% or more, and to anneal to the target sequence at temperatures about 68°-72° C. Any stretch of nucleotides which would result in hairpin structures and primer-primer dimerizations is avoided.

Original or selected cDNA libraries or a human genomic library are used to extend the sequence; the latter is most useful to obtain 5′ upstream regions. If more extension is necessary or desired, additional sets of primers are designed to further extend the known region.

By following the instructions for the XL-PCR kit (Perkin Elmer) and thoroughly mixing the enzyme and reaction mix, high fidelity amplification is obtained. Beginning with 40 pmol of each primer and the recommended concentrations of all other components of the kit, PCR is performed using the Peltier Thermal Cycler (PTC200; MJ Research, Watertown Mass.) and the following parameters:

| | |
|---|---|
| Step 1 | 94° C. for 1 min (initial denaturation) |
| Step 2 | 65° C. for 1 min |
| Step 3 | 68° C. for 6 min |
| Step 4 | 94° C. for 15 sec |
| Step 5 | 65° C. for 1 min |
| Step 6 | 68° C. for 7 min |
| Step 7 | Repeat step 4-6 for 15 additional cycles |
| Step 8 | 94° C. for 15 sec |
| Step 9 | 65° C. for 1 min |
| Step 10 | 68° C. for 7:15 min |
| Step 11 | Repeat step 8-10 for 12 cycles |
| Step 12 | 72° C. for 8 min |
| Step 13 | 4° C. (and holding) |

A 5-10 μl aliquot of the reaction mixture is analyzed by electrophoresis on a low concentration (about 0.6-0.8%) agarose mini-gel to determine which reactions were successful in extending the sequence. Bands thought to contain the largest products were selected and cut out of the gel. Further purification involves using a commercial gel extraction method such as QIAQUICK (QIAGEN Inc). After recovery of the DNA, Klenow enzyme was used to trim single-stranded, nucleotide overhangs creating blunt ends which facilitate religation and cloning.

After ethanol precipitation, the products are redissolved in 13 μl of ligation buffer, 1 μl T4-DNA ligase (15 units) and 1 μl T4 polynucleotide kinase are added, and the mixture is incubated at room temperature for 2-3 hours or overnight at 16° C. Competent E. coli cells (in 40 μl of appropriate media) are transformed with 3 μl of ligation mixture and cultured in 80 μl of SOC medium (Sambrook J et al, supra). After incubation for one hour at 37° C., the whole transformation mixture is plated on Luria Bertani (LB)-agar (Sambrook J et al, supra) containing 2xCarb. The following day, several colonies are randomly picked from each plate and cultured in 150 μl of liquid LB/2xCarb medium placed in an individual well of an appropriate, commercially-available, sterile 96-well microtiter plate. The following day, 5 μl of each overnight culture is transferred into a non-sterile 96-well plate and after dilution 1:10 with water, 5 μl of each sample is transferred into a PCR array.

For PCR amplification, 18 μl of concentrated PCR reaction mix (3.3x) containing 4 units of rTth DNA polymerase, a vector primer and one or both of the gene specific primers used for the extension reaction are added to each well. Amplification is performed using the following conditions:

| | |
|---|---|
| Step 1 | 94° C. for 60 sec |
| Step 2 | 94° C. for 20 sec |
| Step 3 | 55° C. for 30 sec |
| Step 4 | 72° C. for 90 sec |
| Step 5 | Repeat steps 2-4 for an additional 29 cycles |
| Step 6 | 72° C. for 180 sec |
| Step 7 | 4° C. (and holding) |

Aliquots of the PCR reactions are run on agarose gels together with molecular weight markers. The sizes of the PCR products are compared to the original partial cDNAs, and appropriate clones are selected, ligated into plasmid and sequenced.

### VI Labeling and Use of Hybridization Probes

Hybridization probes derived from SEQ ID NO:2 are employed to screen cDNAs, genomic DNAs or mRNAs. Although the labeling of oligonucleotides, consisting of about 20 base-pairs, is specifically described, essentially the same procedure is used with larger cDNA fragments. Oligonucleotides are designed using state-of-the-art software such as OLIGO 4.06 (National Biosciences), labeled by combining 50 pmol of each oligomer and 250 mCi of [Υ-³²p] adenosine triphosphate (Amersham, Chicago Ill.) and T4 polynucleotide kinase (DuPont NEN, Boston Mass.). The labeled oligonucleotides are substantially purified with Sephadex G-25 super fine resin column (Pharmacia). A portion containing 10⁷ counts per minute of each of the sense and antisense oligonucleotides is used in a typical membrane based hybridization analysis of human genomic DNA digested with one of the following endonucleases (Ase I, Bgl II, Eco RI, Pst I, Xba I, or Pvu II; DuPont NEN).

The DNA from each digest is fractionated on a 0.7 percent agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under increasingly stringent conditions up to 0.1 ×saline sodium citrate and 0.5% sodium dodecyl sulfate. After XOMAT AR film (Kodak, Rochester N.Y.) is exposed to the blots in a Phosphoimager cassette (Molecular Dynamics,-Sunnyvale Calif.) for several hours, hybridization patterns are compared visually.

### VII Antisense Molecules

Although use of antisense oligonucleotides, comprising about 20 base-pairs, is specifically described, essentially the same procedure is used with larger cDNA fragments encoding MCPP. An oligonucleotide designed from SEQ ID NO:2 is used to inhibit expression of naturally occurring MCPP. The complementary oligonucleotide represents the most unique 5′ sequence as shown in FIG. 1A and is used to inhibit transcription by preventing promoter binding to the upstream nontranslated sequence. Using an appropriate portion of the leader and 5′ sequence of SEQ ID NO:2, an effective antisense oligonucleotide includes any 15-20 nucleotides spanning the region which translates into the signal or early coding sequence of the polypeptide as shown in FIGS. 1A and 1B.

### VIII Expression of MCPP

Expression of the MCPP is accomplished by subcloning the cDNAs into appropriate vectors and transfecting the vectors into host cells. In this case, the cloning vector, PSPORT, previously used for the generation of the cDNA library is used to express MCPP in E. coli. Upstream of the cloning site, this vector contains a promoter for β-galactosidase, followed by sequence containing the amino-terminal Met and the subsequent 7 residues of β-galactosidase. Immediately following these eight residues is a bacteriophage promoter useful for transcription and a linker containing a number of unique restriction sites.

Induction of an isolated, transfected bacterial strain with IPTG using standard methods produces a fusion protein which consists of the first seven residues of β-galactosidase, about 5 to 15 residues of linker, and the full length MCPP. The signal sequence directs the secretion of MCPP into the bacterial growth media which can be used directly in the following assay for activity.

### IX MCPP Activity

Chemokine chemotactic activity is measured in 48-well microchemotaxis chambers. In each well, two compartments are separated by a filter that allows the passage of cells from one compartment into the other in response to a chemical gradient. Cell culture medium into which MCPP has been secreted is placed on one side of a polycarbonate filter, and peripheral blood cells are suspended in the same media opposite side of the filter. Sufficient incubation time is allowed for the cells to traverse the filter in response to diffusion and resulting concentration gradient of MCPP. Filters are recovered from each well, and specific cell types, eg, monocytes, adhering to the side of the filter facing the chemokine are identified and counted.

Specificity of the chemoattraction is determined by performing the assay on fractionated populations of cells such as enriched populations of neutrophils, mononuclear cells, monocytes or lymphocytes obtained by density gradient centrifugation. Specific T cell populations can be purified using CD8+ and CD4+ specific antibodies for negative selection.

### X Production of MCPP Specific Antibodies

MCPP substantially purified using PAGE electrophoresis (Sambrook, supra) is used to immunize rabbits and to produce antibodies using standard protocols. The amino acid sequence translated from MCPP is analyzed using DNASTAR software (DNAStar Inc) to determine regions of high antigenicity and a corresponding oligopolypeptide is synthesized and used to raise antibodies by means known to those of skill in the art. Analysis to select appropriate epitopes, such as those near the C-terminus or in hydrophilic regions (shown in FIGS. 4 and 5) is described by Ausubel F M et al (supra).

Typically, the oligopeptides are 15 residues in length, synthesized using an Applied Biosystems Peptide Synthesizer Model 431A using fmoc-chemistry, and coupled to keyhole limpet hemocyanin (KLH, Sigma) by reaction with M-maleimidobenzoyl-N-hydroxysuccinimide ester (MBS; Ausubel F M et al, supra). Rabbits are immunized with the oligopeptide-KLH complex in complete Freund's adjuvant. The resulting antisera are tested for antipeptide activity, for example, by binding the peptide to plastic, blocking with 1% BSA, reacting with rabbit antisera, washing, and reacting with radioiodinated, goat anti-rabbit IgG.

### XI Purification of Naturally Occurring MCPP Using Specific Antibodies

Naturally occurring or recombinant MCPP is substantially purified by immunoaffinity chromatography using antibodies specific for MCPP. An immunoaffinity column is constructed by covalently coupling MCPP antibody to an activated chromatographic resin such as CnBr-activated Sepharose (Pharmacia Biotech). After the coupling, the resin is blocked and washed according to the manufacturer's instructions.

Media containing MCPP is passed over the immunoaffinity column, and the column is washed under conditions that allow the preferential absorbance of MCPP (eg, high ionic strength buffers in the presence of detergent). The column is eluted under conditions that disrupt antibody/MCPP binding (eg, a buffer of pH 2-3 or a high concentration of a chaotrope such as urea or thiocyanate ion), and MCPP is collected.

### XII Identification of Molecules Which Interact with MCPP

MCPP, or biologically active fragments thereof, are labelled with ¹²⁵I Bolton-Hunter reagent (Bolton, A E and Hunter, W M (1973) Biochem J 133:529-539). Candidate molecules previously arrayed in the wells of a 96 well plate are incubated with the labelled MCPP, washed and any wells with labelled MCPP complex are assayed. Data obtained using different concentrations of MCPP are used to calculate values for the number, affinity, and association of MCPP with the candidate molecules.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

What is claimed is:

1. An isolated polypeptide selected from the group consisting of:
a) a polypeptide comprising the amino acid sequence of SEQ ID NO:1,
b) a polypeptide comprising a naturally occurring amino acid sequence at least 90% identical to the amino acid sequence of SEQ ID NO:1,
c) a biologically active fragment of a polypeptide having the amino acid sequence of SEQ ID NO:1, and
d) an immunogenic fragment of a polypeptide having the amino acid sequence of SEQ ID NO:1.

2. An isolated polypeptide of claim 1 comprising the amino acid sequence of SEQ ID NO:1.

3. An isolated polynucleotide encoding a polypeptide of claim 1.

4. An isolated polynucleotide encoding a polypeptide of claim 2.

5. An isolated polynucleotide of claim 4 comprising the polynucleotide sequence of SEQ ID NO:2.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method of producing a polypeptide encoded by a polynucleotide of claim 3, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide of claim 3, and
b) recovering the polypeptide so expressed.

10. A method of claim 9, wherein the polypeptide comprises the amino acid sequence of SEQ ID NO:1.

11. An isolated antibody which specifically binds to a polypeptide of claim 1.

12. An isolated polynucleotide selected from the group consisting of:
a) a polynucleotide comprising the polynucleotide sequence of SEQ ID NO:2,
b) a polynucleotide comprising a naturally occurring polynucleotide sequence at least 90% identical to the polynucleotide sequence of SEQ ID NO:2,
c) a polynucleotide complementary to a polynucleotide of a), d) a polynucleotide complementary to a polynucleotide of b), and e) an RNA equivalent of a)-d).

13. An isolated polynucleotide comprising at least 60 contiguous nucleotides of a polynucleotide of claim 12.

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. A method of claim 14, wherein the probe comprises at least 60 contiguous nucleotides.

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises the amino acid sequence of SEQ ID NO:1.

19. A method for treating a disease or condition associated with decreased expression of functional MCPP, comprising administering to a patient in need of such treatment the composition of claim 17.

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. A composition comprising an agonist compound identified by a method of claim 20 and a pharmaceutically acceptable excipient.

22. A method for treating a disease or condition associated with decreased expression of functional MCPP, comprising administering to a patient in need of such treatment a composition of claim 21.

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. A composition comprising an antagonist compound identified by a method of claim 23 and a pharmaceutically acceptable excipient.

25. A method for treating a disease or condition associated with overexpression of functional MCPP, comprising administering to a patient in need of such treatment a composition of claim 24.

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. A method of screening for a compound that modulates the activity of the polypeptide of claim 1, the method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30. A diagnostic test for a condition or disease associated with the expression of MCPP in a biological sample, the method comprising:
a) combining the biological sample with an antibody of claim 11, under conditions suitable for the antibody to bind the polypeptide and form an antibody:polypeptide complex, and
b) detecting the complex, wherein the presence of the complex correlates with the presence of the polypeptide in the biological sample.

31. The antibody of claim 11, wherein the antibody is:
a) a chimeric antibody,
b) a single chain antibody,
c) a Fab fragment,
d) a F(ab′)₂ fragment, or
e) a humanized antibody.

32. A composition comprising an antibody of claim 11 and an acceptable excipient.

33. A method of diagnosing a condition or disease associated with the expression of MCPP in a subject, comprising administering to said subject an effective amount of the composition of claim 32.

34. A composition of claim 32, wherein the antibody is labeled.

35. A method of diagnosing a condition or disease associated with the expression of MCPP in a subject, comprising administering to said subject an effective amount of the composition of claim 34.

36. A method of preparing a polyclonal antibody with the specificity of the antibody of claim 11, the method comprising:
a) immunizing an animal with a polypeptide consisting of the amino acid sequence of SEQ ID NO:1, or an immunogenic fragment thereof, under conditions to elicit an antibody response,
b) isolating antibodies from said animal, and
c) screening the isolated antibodies with the polypeptide, thereby identifying a polyclonal antibody which binds specifically to a polypeptide comprising the amino acid sequence of SEQ ID NO:1.

37. A polyclonal antibody produced by a method of claim 36.

38. A composition comprising the polyclonal antibody of claim 37 and a suitable carrier.

39. A method of making a monoclonal antibody with the specificity of the antibody of claim 11, the method comprising:
a) immunizing an animal with a polypeptide consisting of the amino acid sequence of SEQ ID NO:1, or an immunogenic fragment thereof, under conditions to elicit an antibody response,
b) isolating antibody producing cells from the animal,
c) fusing the antibody producing cells with immortalized cells to form monoclonal antibody-producing hybridoma cells,
d) culturing the hybridoma cells, and
e) isolating from the culture monoclonal antibody which binds specifically to a polypeptide comprising the amino acid sequence of SEQ ID NO:1.

40. A monoclonal antibody produced by a method of claim 39.

41. A composition comprising the monoclonal antibody of claim 40 and a suitable carrier.

42. The antibody of claim 11, wherein the antibody is produced by screening a Fab expression library.

43. The antibody of claim 11, wherein the antibody is produced by screening a recombinant immunoglobulin library.

44. A method of detecting a polypeptide comprising the amino acid sequence of SEQ ID NO:1 in a sample, the method comprising:
a) incubating the antibody of claim 11 with a sample under conditions to allow specific binding of the antibody and the polypeptide, and
b) detecting specific binding, wherein specific binding indicates the presence of a polypeptide comprising the amino acid sequence of SEQ ID NO:1 in the sample.

45. A method of purifying a polypeptide comprising the amino acid sequence of SEQ ID NO:1 from a sample, the method comprising:
a) incubating the antibody of claim 11 with a sample under conditions to allow specific binding of the antibody and the polypeptide, and
b) separating the antibody from the sample and obtaining the purified polypeptide comprising the amino acid sequence of SEQ ID NO:1.

46. A microarray wherein at least one element of the microarray is a polynucleotide of claim 13.

47. A method of generating an expression profile of a sample which contains polynucleotides, the method comprising:
a) labeling the polynucleotides of the sample,
b) contacting the elements of the microarray of claim 46 with the labeled polynucleotides of the sample under conditions suitable for the formation of a hybridization complex, and
c) quantifying the expression of the polynucleotides in the sample.

48. An array comprising different nucleotide molecules affixed in distinct physical locations on a solid substrate, wherein at least one of said nucleotide molecules comprises a first oligonucleotide or polynucleotide sequence specifically hybridizable with at least 30 contiguous nucleotides of a target polynucleotide, and wherein said target polynucleotide is a polynucleotide of claim 12.

49. An array of claim 48, wherein said first oligonucleotide or polynucleotide sequence is completely complementary to at least 30 contiguous nucleotides of said target polynucleotide.

50. An array of claim 48, wherein said first oligonucleotide or polynucleotide sequence is completely complementary to at least 60 contiguous nucleotides of said target polynucleotide.

51. An array of claim 48, wherein said first oligonucleotide or polynucleotide sequence is completely complementary to said target polynucleotide.

52. An array of claim 48, which is a microarray.

53. An array of claim 48, further comprising said target polynucleotide hybridized to a nucleotide molecule comprising said first oligonucleotide or polynucleotide sequence.

54. An array of claim 48, wherein a linker joins at least one of said nucleotide molecules to said solid substrate.

55. An array of claim 48, wherein each distinct physical location on the substrate contains multiple nucleotide molecules, and the multiple nucleotide molecules at any single distinct physical location have the same sequence, and each distinct physical location on the substrate contains nucleotide molecules having a sequence which differs from the sequence of nucleotide molecules at another distinct physical location on the substrate.

56. A polypeptide of claim 1, comprising the amino acid sequence of SEQ ID NO:1.

57. A polynucleotide of claim 12, comprising the polynucleotide sequence of SEQ ID NO:2.


SEQUENCE LISTING (7 sequences)

SEQ ID NO: 1 - protein, 109 aa, Homo sapiens
  misc_feature: Incyte ID No 965517CD1
MLKLTPLPSKMKVSAALLCLLLMAATFSPQGLAQPDSVSIPITCCFNVINRKIPIQRLESYTRITNIQCPKEAVIFKTKRGKEVCADPKERWVRDSMKHLDQIFQNLKP

SEQ ID NO: 2 - DNA, 856 bp, Homo sapiens
  misc_feature: Incyte ID No 965517CB1
aaaccttcacctctcatgctgaagctcacacccttgccctccaagatgaaggtttctgcagcgcttctgtgcctgctgctcatggcagccactttcagccctcagggacttgctcagccagattcagtttccattccaatcacctgctgctttaacgtgatcaataggaaaattcctatccagaggctggagagctacacaagaatcaccaacatccaatgtcccaaggaagctgtgatcttcaagaccaaacggggcaaggaggtctgtgctgaccccaaggagagatgggtcagggattccatgaagcatctggaccaaatatttcaaaatctgaagccatgagccttcatacatggactgagagtcagagcttgaagaaaagcttatttattttccccaacctcccccaggtgcagtgtgacattattttattataacatccacaaagagattatttttaaataatttaaagcataatatttcttaaaaagtatttaattatatttaagttgttgatgttttaactctatctgtcatacatcctagtgaatgtaaaatgcaaaatcctggtgatgtgttttttgtttttgttttcctgtgagctcaactaagttcacggccaaangtcattgttctccctcctaccngtncgtagtgttgtggggtcctcccntggatcatcaaggtgaaacacttaggtattctttggcaatcagtgctcctgtaagtcaaatgtgtgctttgtactgctgttgttgaaattgangttactgtanataactatggaattttgaaaaaaaatttcaaaaagaaaaanatatatataatttaaaactaaaaaaaaaaaaaaaaa

SEQ ID NO: 3 - protein, 109 aa, Homo sapiens
  misc_feature: GenBank ID No g288397
MWKPMPSPSNMKASAALLCLLLTAAAFSPQGLAQPVGINTSTTCCYRFINKKIPKQRLESYRRTTSSHCPREAVIFKTKLDKEICADPTQKWVQDFMKHLDKKTQTPKL

SEQ ID NO: 4 - protein, 99 aa, Homo sapiens
  misc_feature: GenBank ID No g338809
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 5 - DNA, 236 bp, Homo sapiens
  misc_feature: Incyte ID No 515733
cagaaaccttcanctctcatgctgaagctcacanccttgccctccaagatgaaggtttctgcagcgttctgtgcctgctgctcatggcagccactttcagccctcagggacttgctcagncagattcagtttccattccaatcanctgctgctttaangtgatcaataggaaaattnctatccagaggctggagagctacanaagaatnaccaacatccaatgtcccaaggaagnt

SEQ ID NO: 6 - DNA, 228 bp, Homo sapiens
  misc_feature: Incyte ID No 518226
ccacgcgtccgccagaaaccttcacctctnatgctgaagctcacanccttgccctccaagatgaaggtttntgcagcgnttctgtgcctgctgntcatggcagccactttcagccctcagggacttgntcagccagattcagtttccattccaatcanctgctgntttaacgtgatcaataggaaaattcctattcagaggntggagagctacacaagantnancaac

SEQ ID NO: 7 - DNA, 256 bp, Homo sapiens
  misc_feature: Incyte ID No 568961
cggnacggtggnccttcacctctcatgctgaagctcacacccttgccctccaagatgaaggtttctgcagcgcttctgtgcctgctgctcatggcagccactttcagccctcagggacttgctcagccagattcagtttccattccaatcacctgctgctttaacgtgatcaataggaaaattcctatccagaggctggagagctacacaagaatcaccaacatccaatgtcccaaggaagctgtgatcttcaaga